(19) United States
(12) Patent Application Publication
(10) Pub. No.: US 2004/0102904 A1
(43) Pub. Date: May 27, 2004

(54) Clinical laboratory quality control randomizer

(76) Inventor: Roger Bennison, Roanoke, VA (US)

Correspondence Address: ROGER BENNISON, 7429 FORT MASON DRIVE, ROANOKE, VA, 24018, US

(21) Appl. No.: 10/718,422

(22) Filed: Nov. 20, 2003

Related U.S. Application Data
(60) Provisional application No. 60/427,724, filed on Nov. 20, 2002.

(51) Int. Cl. G06F 19/00; G01N 33/48; G01N 33/50

(52) U.S. Cl. 702/19

(57) ABSTRACT

A system and method of organizing the routine quality control tests performed in a clinical laboratory such that verification of the analytical machines is based on sound statistical principles. A computer-based program which requests that information be entered by an operator, then supplies the necessary guidance to locate the control tests in position and time to assure statistical validity. A data-management tool to facilitate compliance with government and professional standards of procedure.

### CROSS-REFERENCE TO RELATED APPLICATIONS

This application claims the benefit of U.S. Disclosure Document No. 482982, filed Nov. 28, 2000, which is hereby incorporated by reference herein in its entirety. This application claims the benefit of provisional Patent Application No. 60/427,724, filed Nov. 20, 2002 which is hereby incorporated by reference herein in its entirety.

### STATEMENT REGARDING FEDERALLY SPONSORED RESEARCH OR DEVELOPMENT

Not Applicable

### REFERENCE TO SEQUENCE LISTING, A TABLE OR A COMPUTER PROGRAM LISTING COMPACT DISK APPENDIX

Not Applicable

### BACKGROUND OF THE INVENTION

The present invention relates generally to a new and improved process and apparatus for managing a clinical laboratory in which analytical tests are performed on samples of human tissue or fluids. Government and professional accreditation bodies require that acceptable quality control procedures be followed in such laboratories, so that the results obtained can be relied upon. These procedures require that control tests be performed periodically, using samples of fluids whose composition is accurately known. Such ‘control’ fluid samples are widely available commercially, and the interpretation of their results employs well-known techniques such as Westgard's multi-rule technique and the use of Levey-Jennings control charts. Control tests are performed by introducing test samples into the regular sequence of patient tests.

The need for the present invention arises because of the arbitrary ways in which the timing and location of control tests are decided upon.

In order that the results of control tests may safely be subjected to statistical analysis, they must be obtained in a manner that avoids bias in the selection of timing or location. Such a technique is lacking in present-day laboratories. It is an objective of the present invention that control tests shall in future be performed in such a way that their results may safely be interpreted by statistical methods.

### BRIEF SUMMARY OF THE INVENTION

The present invention is a system and method for managing and implementing clinical laboratory tests. In one embodiment of the invention, a computer program facilitates the employment of the preferred techniques. By following the methods of the present invention, laboratory technicians will comply with the statistical rules governing the validity of test results, and also will comply with the performance requirements of various government and professional oversight bodies. The laboratory technician specifies the parameters of his or her testing schedule, such as the duration of the time interval during which tests will be performed and the number of patient samples to be handled by the laboratory. The invention provides the technician with direction as to the timing and placement of control test samples within sequences of patient samples, randomly allocating their physical location and temporal distribution. The technique of random allocation satisfies the requirement that no bias as to time or position is possible, as required by the mathematical rules of statistical analysis. The preferred method is adaptable; it can be used with multiple testing machines, and in situations where mandatory repeat tests are required.

### BRIEF DESCRIPTION OF THE SEVERAL VIEWS OF THE DRAWINGS

FIG. 1 shows the first screen seen by a user after the computer program is started. It may be seen that the user can proceed by selecting one of the action choices represented by ‘button’ images. This is a well-known method of providing user interactivity with a computer program.

FIG. 2 shows the screen displayed if ‘Begin QC Tasks’ is the chosen button. The user cannot make use of the secured parts of the program until he or she has entered a valid user name and password here.

FIG. 3 offers the user three primary choices of function, positioner, results data entry, or a test random repeat function. Secondary choices allow the editing of information pertaining to operators, instruments or the data storage files.

FIG. 4 shows the screen resulting from the ‘QC Positioner’ choice. The user can choose from descriptive information previously entered to define the present circumstances.

FIG. 5 shows how the screen seen in FIG. 4 changes when the decision is made to randomize the times of tests. The end time of the present work schedule can be defined to make such randomization valid.

### DETAILED DESCRIPTION OF THE INVENTION

In its simplest form, the present invention may be implemented through the use of a pen and sheets of paper for calculation, implementation and recording of the tasks required. In practical terms, such an implementation would place an unacceptable burden on the user. With the present state of the art, however, these functions may be advantageously performed by modern office equipment. In a preferred embodiment of the present invention, the laboratory technician employs a computer system equipped with an application program designed to implement the method of managing control tests.

The program functions as an interactive guide for the user. The laboratory technician or user supplies information about the tasks to be scheduled and is advised as to when and where to place the control samples in the analysis machine.

The method requires that the present parameters of the laboratory and testing machine be known. To ensure that all requisite details are available, the method embodies a mechanism which ‘prompts’ the user to define parameters relevant to later processing and analysis. In one embodiment of the invention, these parameters are:

Operator Name (confirmed by security password entry)

Identification of the machine which will be analyzing the fluid test sample

Identification of the fluid test sample

Definition of the capacity of the machine in terms of the number of distinct and separate locations within the machine wherein the test sample may be placed.

Definition of the time period within which the test must be performed.

Once the above information has been acquired, the method employs known mathematical methods to randomly allocate a location and time at which the test will be performed. The random selection technique confines itself to the defined ranges of time and location when specifying the response. The informational response is displayed for the user, and may also be provided on a paper printout to facilitate the activities of the laboratory.

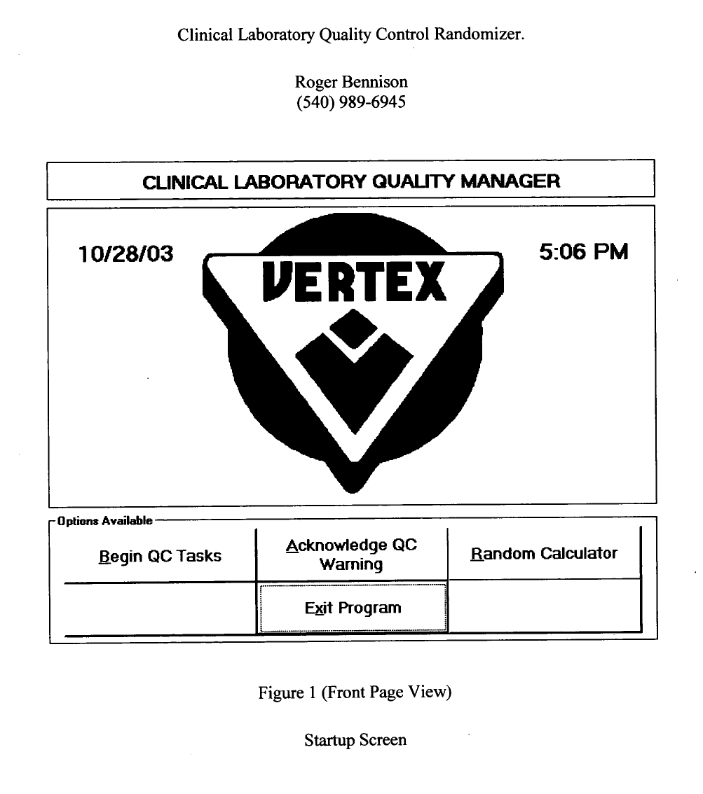

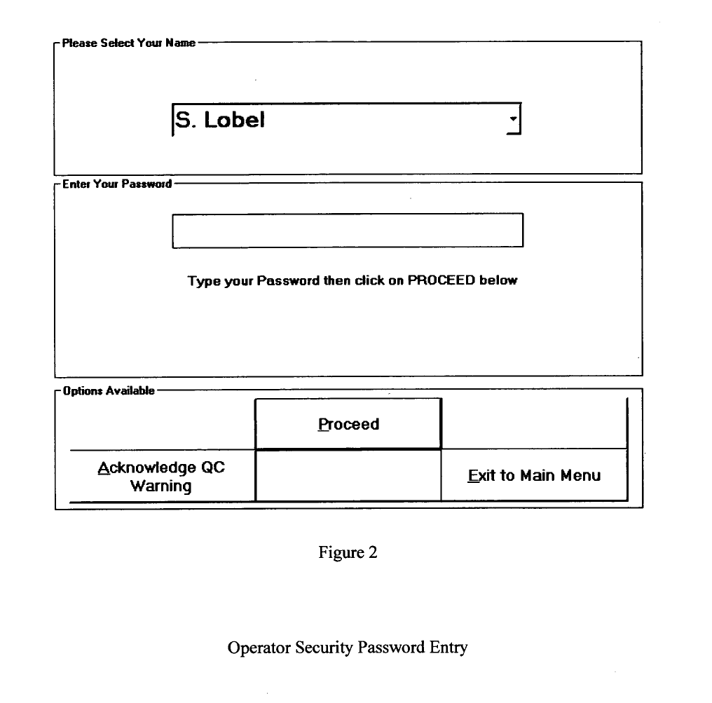

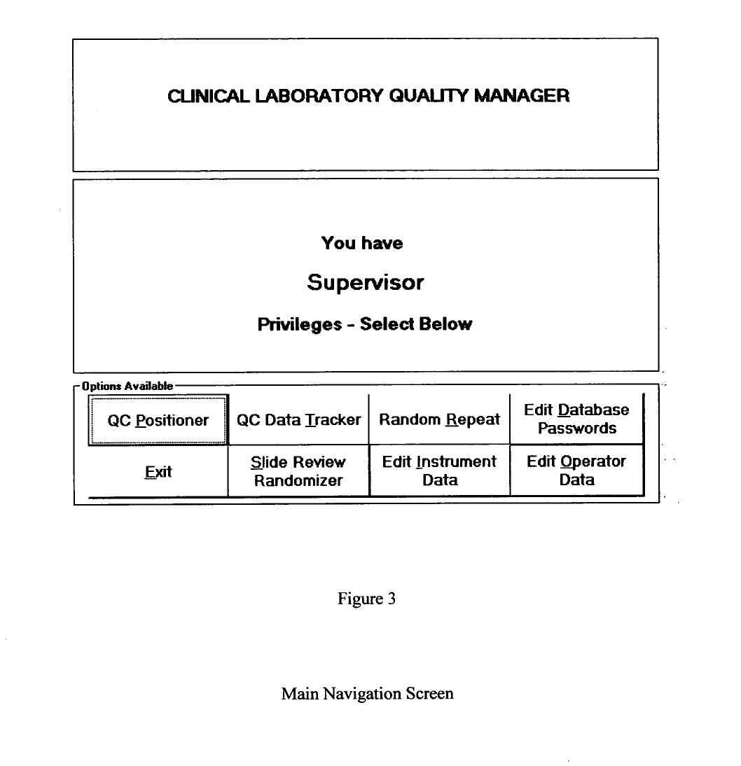

The computer program shows the user a ‘startup screen’ (FIG. 1), from which they can choose to begin the procedure for scheduling a control test. After entering a user name and password, the program presents a screen on which the technician can choose one of three functions to perform (FIG. 2). For the purposes of this description, the primary functions ‘QC Positioner’ and ‘QC Data Tracker’ will be detailed.

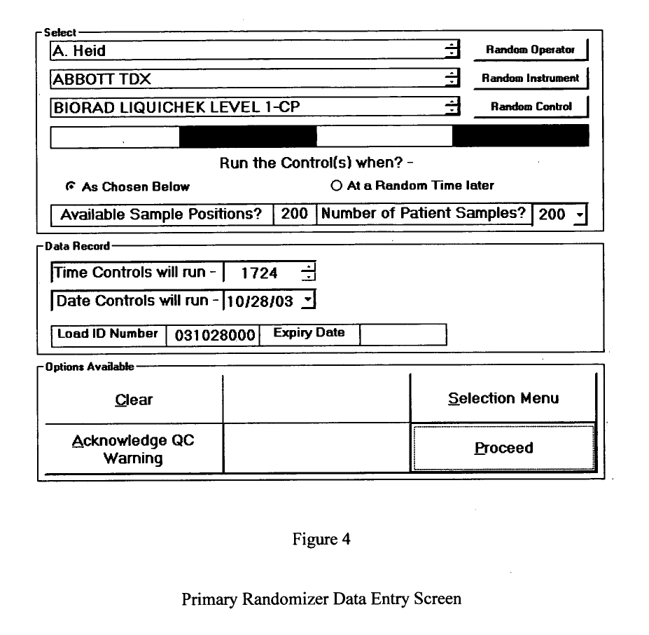

After the user has selected ‘QC Positioner’, the screen changes to prompt the user for required information about the current operating parameters (FIG. 4). Typical parameters are: Operator Name, Testing Machine Identification, Control Test Identification, Number of possible locations on the machine, Number of Patient samples to be loaded along with the control test sample.

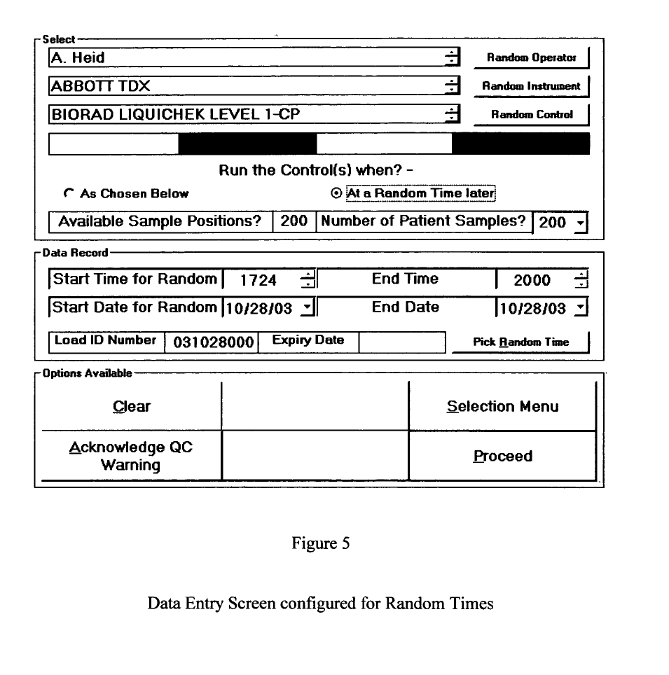

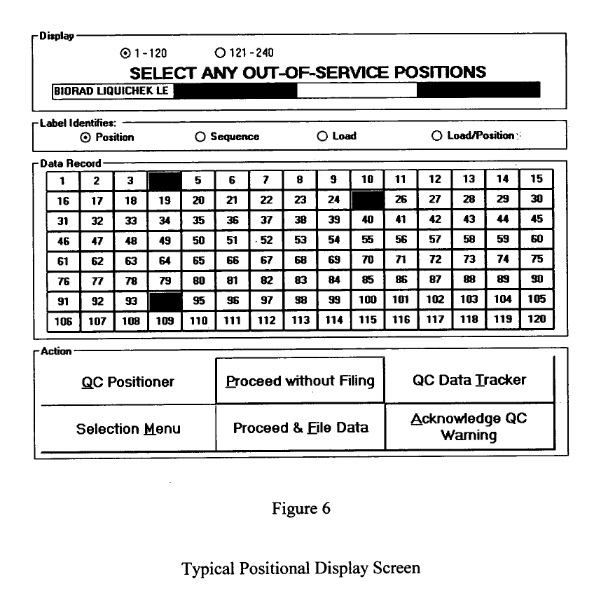

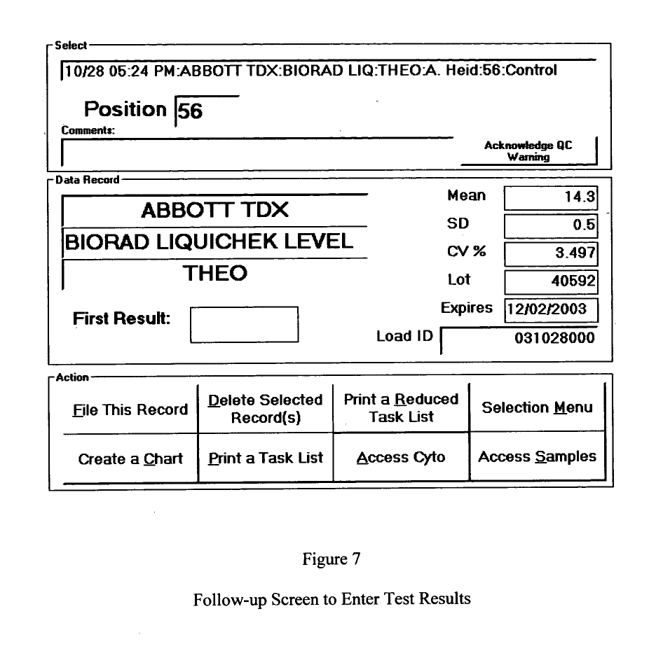

If the user wishes to invoke the randomization of test times, the necessary start and end times for the working period are entered on the secondary version of the screen (FIG. 5).

After the user has specified the necessary parameters and chosen to proceed, the screen of FIG. 6 is illustrative of the position display. As seen in FIG. 6, the user is being offered the option to define certain machine test locations as ‘out of service’, before proceeding to allocate the control test randomly to one of the remaining locations.

Once the user has placed the test sample or samples as directed, the analysis machine is allowed to complete its work, and the test result is entered into the data storage space of the computer. FIG. 7 shows the screen made available by selecting ‘QC Data Tracker’ from the screen shown in FIG. 2. Paragraph 15 above refers.

What I claim as my invention is:

1. A system and method of controlling the activities of a clinical laboratory in such a way that the results of quality control tests may be accepted as statistically valid, and not subject to bias.

2. The system and method according to claim 1, wherein the functions of the system are performed by a computer program operating on a computer.

3. The system and method according to claim 2, wherein the user can define the present operating parameters of the laboratory by entering data at the computer keyboard.

4. The system and method according to claim 3, wherein the user can define the present operating parameters of the laboratory by selecting data from a previously filled database of typical parameter values.

5. The system and method according to claim 4, wherein the location of control test samples within an analytical machine is specified randomly within the range of possible locations.

6. The system and method according to claim 5, wherein the time at which control test samples are to be analyzed by the machine is specified randomly within the range of possible times.

7. The system and method according to claim 6, wherein the test data resulting from the test or tests can be entered and recorded in a storage database.

8. The system and method according to claim 7, wherein the test data resulting from the test or tests can be displayed graphically for user evaluation.

9. The system and method according to claim 6, wherein the test data resulting from the test or tests and recorded in a storage database can be printed for user evaluation.

10. The system and method according to claim 6, wherein the test data resulting from the test or tests are received directly from the analytical machine via a means of communication and recorded in a storage database.

11. The system and method according to claim 6, wherein the parameters of the forthcoming tests are passed on to other laboratory information systems or machines via a means of communication and are recorded in a storage database.

12. The system and method according to claim 6, wherein the repeat testing of patient samples is facilitated by the provision of a parameter entry screen appropriate to that task.
